(19) United States
(12) Patent Application Publication
(10) Pub. No.: US 2007/0110804 A1
(43) Pub. Date: May 17, 2007

(54) Drug polymer complexes

(75) Inventor: Garfield P. Royer, Frederick, MD (US)

Correspondence Address: NIXON & VANDERHYE, PC, 901 NORTH GLEBE ROAD, 11TH FLOOR, ARLINGTON, VA, 22203, US

(73) Assignee: ROYER BIOMEDICAL, INC., FREDERICK, MD (US)

(21) Appl. No.: 10/561,245

(22) PCT Filed: Jun. 18, 2004
(86) PCT No.: PCT/US04/19236

§ 371 Date: Dec. 19, 2005

Related U.S. Application Data
(60) Provisional application No. 60/479,840, filed on Jun. 20, 2003.

(51) Int. Cl. A61K 38/14; A61K 31/7048; A61K 31/7034; A61K 31/5415; A61K 31/485; A61K 31/135; A61K 31/137; A61K 9/22; A61K 31/445; A61K 31/24

(52) U.S. Cl. 424/468; 514/8; 514/35; 514/28; 514/536; 514/282; 514/304; 514/225.5; 514/225.8; 514/317; 514/649

(57) ABSTRACT

This invention relates to polymeric complexes of drugs to be employed as (or in) sustained release-formulations comprising a cationic active agent, and a polyanionic water soluble complexing polymer of sufficient molecular weight that it forms a gel when mixed with said active agent. The invention also relates to the manufacture of such sustained release compositions and their many uses. Also included is a molded prosthesis comprising a prosthesis including a sustained release composition comprising a cationic anti-infective and a complexing polymer.

### FIELD OF THE INVENTION

This invention relates generally to the production and use of drug polymer complexes. The complexes are resorbable. Sustained and/or controlled release of medicinal agents and other bioactive substances are the primary uses of these systems.

### BACKGROUND OF THE INVENTION

Polymer matrices designed for controlled release of bioactive compounds can be non-resorbable or resorbable. In general, resorbable means degradable in the body by erosion from the surface or breakdown from within. The mechanism can involve either a chemical reaction, such as hydrolysis, or dissolution.

Non-resorbable polymers, such as polymethylnethacrylate, have been used for antibiotic delivery. These materials suffer from the disadvantage that they must be retrieved, which involves a second intervention and entails the risk of infection (H W Bucholz, et al., (1970) Chiburg, 43, 446).

Resorbable polymer matrices for controlled release are usually based on an oxygen-containing monomer, which is condensed in organic solvent to yield the polymeric product. The bioactive agent and the polymer are then combined in such a way as to give a timed-release formulation. The combination of active ingredient and polymer often involves organic solvents as well. The use of organic solvents is a decided disadvantage, especially when large-scale production is required. Toxic residues of organic solvents are a concern. Proteins and many polypeptides are incompatible with organic solvents.

The types of polymers in this category include:
polyesters
polyanhydrides
polyketals
poly(orthoesters)
polyurethanes
   (Burkersroda, F V and Goepferich, A M in Biomedical Materials, T Neenan, M Marcolongo and R F Valentini, eds. (1999), page 23, Materials Research Society, Warrendale Pa.).

Naturally occurring proteins may be used as structural components in drug-delivery matrices (Royer, U.S. Pat. No. 4,349,530; Royer, U.S. Pat. No. 5,783,214; Lee et al, Science (1981) 233-235). One deficiency of proteinaceous delivery matrices is that they can exhibit instability especially in environments where an inflammatory reaction is present such as a site of localized sepsis.

Commonly owned WO 99/15150 and U.S. Pat. No. 6,391,336 disclose stable, yet practical compositions for use in inflamed sites comprising an inorganic compound, a matrix polymer and/or a complexing agent. This composition has the advantage of being biocompatible but, unlike synthetic organic polymers, no non-aqueous solvents are required in the preparation. The drug is incorporated as a solid or as part of the matrix polymer solution. The material can also be used as a cement, that is, it can be injected directly into a lesion and allowed to solidify in situ.

Commonly owned U.S. Pat. No. 6,497,901 discloses a delivery system with a conditioning agent.

U.S. Pat. No. 5,716,631 relates to long acting narcotic compositions comprising a water-soluble analgesic or antagonist drug dispersed within a polymer matrix, methods of producing the same and treatments with the soluble complex.

### OBJECTS OF THE INVENTION

It is an object of this invention to provide a safe resorbable delivery system that can be designed and fashioned to provide controlled release of bioactive substances over a pre-determined time-course.

It is an object of this invention to improve control of medicinal release rate and residence time.

### SUMMARY OF THE INVENTION

The subject invention relates to compositions for the controlled release of an active agent comprising a cationic active agent, and a polyanionic water-soluble complexing polymer of sufficient molecular weight that it forms a gel when mixed with said active agent.

The invention also relates to methods of obtaining sustained release of medicinals and other active agents, including treating an infection in a mammal comprising administering to said mammal a sustained release composition comprising a cationic anti-infective and a polyanionic water soluble complexing polymer of sufficient molecular weight that it forms a gel with said anti-infective.

Also included is a method of regionally blocking nerves or systemically treating pain in a mammal comprising administering by injection to said mammal a composition comprising an anesthetic or analgesic and a complexing polymer.

The invention also includes a molded prosthesis comprising a prosthesis including a sustained release composition comprising a cationic anti-infective and a polyanionic water soluble complexing polymer.

Also taught by the invention is a method of producing a sustained release gel composition comprising mixing a cationic active agent and a polyanionic water soluble complexing polymer. These complexes can deliver drugs locally or can be employed as depots for systemic delivery.

### BRIEF DESCRIPTION OF THE DRAWINGS

FIG. 1 shows release profiles of dextran sulfate complexes of vancomycin and amikacin.

FIG. 2 shows a release profile of dextran sulfate complex of methadone.

FIG. 3 shows a release profile of dextran sulfate complex of tetracaine.

FIG. 4 shows a release profile of dextran sulfate complex of chlorpromazine.

FIG. 5 shows a release profile of dextran sulfate complex of apomorphine.

FIG. 6 shows release profiles of two oxycodone complexes made with different dextran sulfates.

### DETAILED DESCRIPTION OF THE INVENTION

This invention relates to the preparation and use of polymeric complexes of drugs to be employed as (or in) sustained release-formulations.

Compositions of the Invention

In an attempt to formulate amikacin in a calcium sulfate matrix for a long lasting drug depot, a poorly soluble polymer-drug complex in the form of a gel was discovered. As part of the normal preparation of a solid dosage form based on calcium sulfate, a solution of amikacin sulfate was mixed with a solution of dextran sulfate (Na). Surprisingly, a clear gelatinous precipitate appeared. It included >90% of the amikacin with <10% remaining in the supernatant. Dextran sulfate (Na) forms a poorly soluble complex when contacted with amikacin sulfate and other cationic antibiotics (see below). The amikacin-dextran sulfate has a low solubility in PBS and releases amikacin in a near zero-order fashion for 40 days in an in vitro assay system.

The release rate in the simplest case is described by
Rate=DA(dC/dx)
D=diffusion coefficient
A=surface area
(dC/dx)=concentration gradient at the device boundary

The diffusion coefficient is dependent on the solubility of the drug, the molecular weight (Mw) of the drug, and the viscosity of the medium (V):
D∝S/vM_{w}

When the active drug is complexed to the polymer, a viscous gel forms; as a consequence, the solubility is decreased, and the viscosity and the apparent molecular weight are increased. As used herein, the term “gel” means the more viscous phase that separates or is separable from the supernatant after the cationic active agent and the polyanionic complexing polymer are mixed. In some cases, supernatant production is minimal.

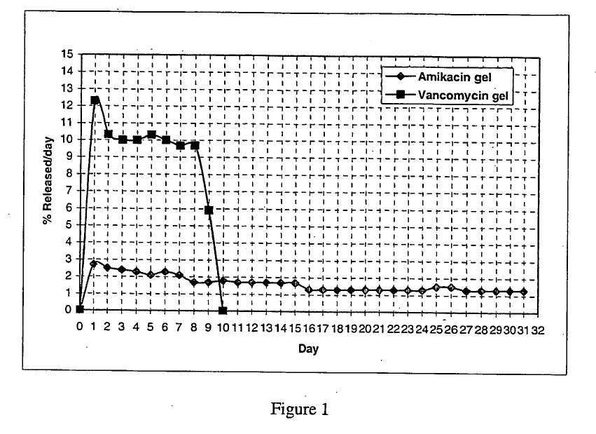

Representative release profiles are shown in FIGS. 1-6. Other compounds, such as clindamycin and various analgesics, have also been successfully complexed as discussed below.

Active Agents

Active agents useful in the subject invention are multidentate cations (at least 2 positive charges), or molecules with a hydrophobic region and an exposed (not buried within the hydrophobic region) cation (typically at an end of the molecule). Cationic peptides can also be formulated according to the invention. Examples are as follows:
Analgesics hydromorphone, oxycodone, morphine, fentanyl, hydrocodone, buprenorphine
Analgesic antagonists methadone, naloxone, naltrexone
Anesthetics dibucaine, tetracaine, procaine, etidocaine, prilocaine, mepivacaine
Anti-infectives amikacin, gentamicin, vancomycin, clindamycin, neomycin, streptomycin, doxycycline, polymyxin B
Anti-tumor agents doxorubicin, procarbazine, bleomycin, vincristine
CNS agents acepromezine, prochlorperazine, clomipramine, ondansetron, sertraline, doxazosine, chlorpromazine, atropine

Additional opioids/analgesics useful in the invention include sufentenil, etorphine, levorphanol, levallorphan, butorphenol, propoxyphene, nalorphine, nalbuphine, nalmefene, codeine, oxymorphone, and dermorphine.

Complexing Polymers

Complexing polymers are water soluble and anionic; they contain pendant groups such as sulfate, carboxylate, phosphate or other negatively charged groups. The complexing polymers are biocompatible and non-toxic. They are of sufficiently high molecular weight that a gel can be prepared with the active agent.

The resulting gel is viscous and often separable from the extraneous aqueous medium. While not wishing to be bound to a particular theory, it is believed that the one polymer chain cross-links to another polymer chain as a consequence of interacting with multiple active agent molecules. In the case of multidentate cations (e.g. amikacin), the crosslinking results from electrostatic interactions between polymer strands. In the case of hydrophobic cations, the interaction of the polymer chains is believed to be hydrophobic in nature. Two or more chains align with the hydrophobic areas in the center of the aggregate to minimize interaction with the polar solvent.

Complexing polymers useful in the subject invention include dextran sulfate, carboxymethylcellulose—CMC-L is low viscosity (50-200 cps, 4%), and CMC-M is medium viscosity (400-800 cps, 2%)—and pentosan sulfate, advantageously of molecular weight greater than 3,800.

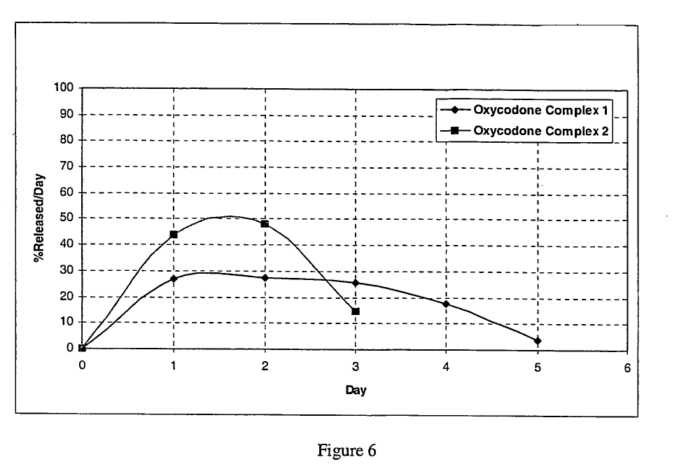

It is possible to alter the release profile by using a lower molecular weight as the complexing polymer. For example with oxycodone, when ½ of the dextran sulfate is lower molecular weight (40,000) and ½ of the dextran sulfate is higher molecular weight (500,000), the release is accelerated (FIG. 6) when compared to all (500,000) dextran sulfate. There are many possible mixtures of complexing polymers (e.g. by varying the molecular weight) that provide the opportunity to tailor the release profile to fit the clinical need.

For an oral capsule of oxycodone, the complexing polymer mixture is advantageously adjusted to give release over a 12-24 hour time span. In contrast, the subcutaneous depot of oxycodone is intended to last days rather than hours, in which case polymers of high molecular weight are used (see Example 17).

Table 1 shows some representative examples using polyanions such as dextran sulfate (Na) and carboxymethylcellulose (Na). All combinations form gelatinous phases where indicated. The solubility and viscosity of the respective gels depend on the active ingredient and the complexing polymer. A “yes” entry means that a complex of low solubility forms on mixing the sodium salt of the polymers and the salt of the active ingredient.

TABLE 1
Representative Polymer/Drug Complexes

| | | Polymer | | |
|---|---|---|---|---|
| Therapeutic | Active | Dextran | | |
| Category | Ingredient | Sulfate-500 | CMC-L | CMC-M |
| Analgesics/ | Hydromorphone | yes | no | no |
| antagonists | Oxycodone | yes | — | — |
| | Methadone | yes | no | no |
| | Naltrexone | yes | no | no |
| | Morphine | yes | — | — |
| | Buprenorphine | yes | — | — |
| Anesthetics | Dibucaine | yes | yes | yes |
| | Tetracaine | yes | yes | yes |
| | Procaine | yes | — | — |
| | Prilocaine | yes | — | — |
| Anti- | Amikacin | yes | no | — |
| infectives | Gentamicin | yes | — | — |
| | Vancomycin | yes | yes | — |
| | Clindamycin | yes | no | no |
| | Doxycycline | yes | yes | yes |
| | Streptomycin | yes | — | — |
| | Oxytetracycline | yes | yes | yes |
| | Neomycin | yes | no | no |
| | Erythromycin | yes | no | no |
| | Tobramycin | yes | — | — |
| Anti-tumor | Doxorubicin | yes | yes | yes |
| agents | | | | |
| CNS agents | Chlorpromazine | yes | yes | yes |
| | Atropine | yes | — | — |
| | Apomorphine | yes | — | — |

Formulations

There are multiple possible dosage forms and applications of these polymer-drug complexes.

Gels

Low viscosity and medium viscosity gels can be made. The solubility and viscosity of the respective gels depend on the active ingredient and the complexing polymer. Some gels are usable as formed, that is, injectable through a needle.

Gums

Calcium sulfate can be added to the gels to form a malleable gum of putty-like consistency, which can be shaped at tableside by the physician. These gums harden and can be used to mold drug-containing implants.

Cements

Cements can be prepared by adding relatively more calcium sulfate-hemihydrate, optionally with calcium stearate. These cements (see e.g. U.S. Pat. No. 6,497,901 hereby incorporated by reference in its entirety) harden to form a material of high compressive strength. Cements can be processed or molded to yield other solid dosage forms such as microgranules, microspheres, 3-mm spheres, bullet-shaped implants and other forms. The cements solidify under water. By adjusting the proportions, the material can be extruded to yield cylinders.

Powders

Dry powders of polymer-drug complexes can be used directly to treat accessible infected sites such as diabetic foot ulcers. This dry polymer-drug complex can be ground and then suspended in various liquid agents for injection. Examples of suspending agents include glycerol, propylene glycol, polyethyleneglycol, and sesame oil.

Dry powders of drug polymer complexes can be finely ground and suspended in a solution of complexing polymer

Combination Products

Class I: Gel 1 (liquid) plus Gel 2 (liquid). In this embodiment one gel product (liquid polymer-drug complex) is mixed with another, either by the manufactured or by the user at the site of administration. An example is amikacin gel plus vancomycin gel. It is well know that these active ingredients act synergistically in treatment of some infections. Another example is amikacin gel plus tetracaine gel for prevention of infection and post-surgical pain control.

Class II: Gel 1 (liquid) plus dry polymer-drug complex. This embodiment can be exemplified by the suspension of dry vancomycin-dextran sulfate in amikacin-dextran sulfate gel.

Suspension Products

Gels containing polymer-drug products can be dried and resuspended in polymer, either by the manufacturer on by the user at the site of administration. An example is dried vancomycin-dextran sulfate complex suspended in either dextran sulfate or CMC. The viscosity of the delivery solution has an influence on the release profile.

Complexed Active Ingredient in Polymer Suspensions

Poorly soluble forms of the active ingredient can be used as the starting material. For example finely-ground enrofloxacin-HCL can be mixed with dextran sulfate solution to provide a sustained-release antibiotic suspension. Free drug can be combined in a fashion to tailor the release profile to meet the clinical need. Other examples of poorly soluble drug complexes include penicillin-procaine, penicillin-benzathin, amikacin-pamoate, and bupivacaine-pamoate. In this embodiment the polymer solution serves as a viscous suspension agent as well as a complexing agent.

Inclusion Products

In this case amikacin (or other multidentate cation) is employed as a cross-linking agent to entrap a neutral molecule. For example, finely-ground ivermectin powder can be suspended in dextran sulfate solution. Addition of amikacin sulfate solution results in a viscous gel. The product is useful as a sustained release injectable for prevention of parasites. Other active ingredients such as paclitaxel and neutral antibiotics can be advantageously formulated using this approach.

### Other Embodiments

The polymer-drug complex in the form of the dry powder can be incorporated into drug delivery systems such as those that include calcium sulfate or other excipients. Polyesters, polyanhydrides, and polyorthoesters are examples of bioerodible polymers, which can be employed. Vinyl polymers such as those used in orthopedic bone cement can be used as well even though these polymers are non-resorbable. Calcium phosphate matrices can be employed. Tricalcium phosphate (e.g. alpha) matrices and hydroxyl-apatite can be mixed with the drug gels to form composites. Gels and powder forms of polymer-drug complexes can be mixed with bone substitutes and grafts for use in fracture repair and filling orthopedic/periodontal defects.

To achieve an initial burst or loading dose, unbound soluble drug can be included in the composition. Various combinations of complexing polymers and drugs can be used to produce long-lasting formulations.

Modes of Administration

Administration of the compositions of the invention can be achieved by injection, surgical implant, oral, i.p., i.a., or topical route. The gel injection can be s.c., i.a., i.m., or i.p. (also true for dried gel suspended in a carrier liquid). Advantageously, the administration is done by parenteral injection.

There are multiple modes of administration for dosage forms related to this invention as illustrated below:

1. Depot/Intra-operative: direct or endoscopic installation

2. Depot: subcutaneous injection

3. Depot: intra-articular injection

4. Depot: subcutaneous, surgical implant

5. Oral: tablet or capsule

6. Transmucosal: buccal or rectal

7. Transdermal: patch or gel

8. Aerosol inhaler

9. Topical (wound dressing)

Some gels can be injected though a needle. Joint sepsis and other localized infections can be thus treated. The gel complex can be subsequently processed to produce other dosage forms as stated earlier. The injectable gel is very convenient because it is easy to administer. It can be injected through a 21-gauge needle or larger.

Uses of the Compositions of the Invention

The compositions of the invention include many types of active agents such as cationic analgesics, analgesic agonists/antagonists, anesthetics, anti-infectives, tranquilizers, cardiovascular drugs, anti-tumor agents, and CNS agents, for a wide variety of uses.

The complex, for example as a viscous gel containing an anti-infective, can be used directly in the body for treating infection, such as joint sepsis. The gel can be subsequently reformulated, either as is or dried. Various anti-infectives useful in conjunction with the formulations of the invention include gentamicin, clarithromycin, azithromycin, flouroquinolone-HCl, doxycycline, minocycline and lincomycin, amikacin, vancomycin, tobramycin, nystatin, and amphotericin B.

Local administration of the compositions of the inventions containing antibiotics is effective is treating orthopedic infections such as joint sepsis and osteomyelitis; other infections such as intra-abdominal abscesses can be addressed in a similar fashion.

Diabetic foot infections are also treatable using a combination such as dried amikacin powder and vancomycin powder. The compositions provide sustained therapeutic levels of antibiotic to the infected site without producing toxic levels systemically.

The compositions of the invention can be used to deliver an anti-infective such as doxycycline to periodontal defects. Immediately after scaling/planning anti-infective gel is applied. The anti-infective compositions are also useful in treating apical root infections.

Prosthetic devices such as orthopedic spacers can be coated with the compositions containing an anti-infective and a complexing polymer to be used in treatment and prevention of infection. Trauma and infected artificial joint prostheses are application areas using this approach.

Doxorubicin and other anti-neoplastic agents can be delivered locally as gels or other dosage forms based on gels as described herein. In one embodiment, localized administration is beneficial in that systemic toxicity is eliminated but concentrations in the area of cancerous tissue are high.

With regard to pain control there are two types of utility. First, is the use of long-lasting local anesthetics for producing regional nerve blocks. The value resides in the alleviation of pain during diagnostic and therapeutic procedures as well as post-surgical pain. Second, chronic pain can be treated using the injectable analgesic gels described herein. Alternatively, oral capsules using polymer complexes with drugs such as oxycodone are of utility for 12-24 hr pain control.

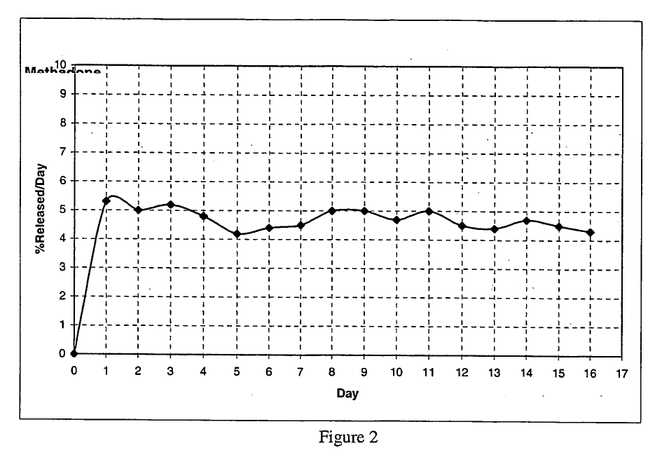

Compositions containing methadone, buprenorphine, naloxone, or naltrexone can be used in the treatment of drug addiction (see FIG. 2 for a release profile of methadone). Rather than employ oral dosages that are issued daily to patient, a longer term treatment with a sustained release injectable is advantageous, especially since the injectable form is not abusable.

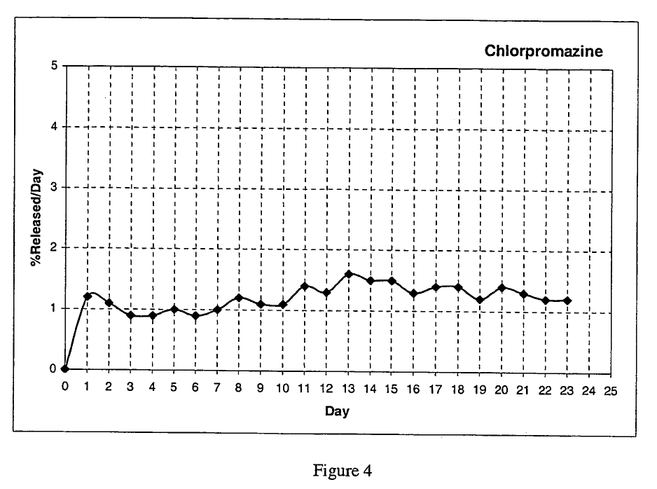

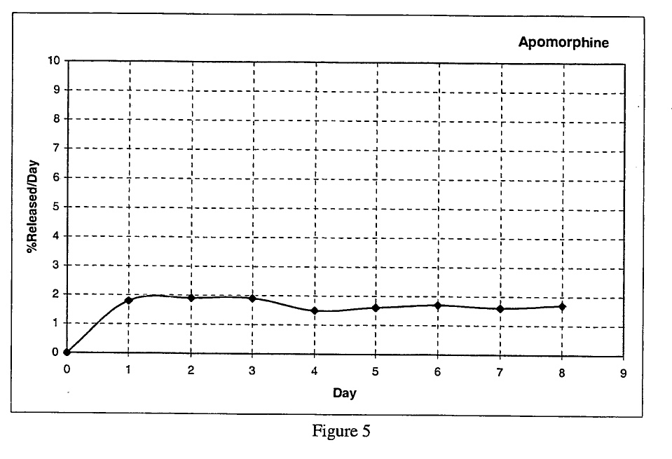

Due to toxicity reduction, patient compliance, and convenience, CNS agents are advantageously delivered using the compositions of the invention. Release profiles of chlorpromazine (anti-psychotic) and apomorphine (anti-parkinsonian) are shown respectively in FIGS. 4 and 5.

Delivery of cells such as mesenchymal stem cells is also possible with the compositions of the subject invention. For example, in the treatment of septic arthritis, mesenchymal stem cells or chondrocytes can be mixed with the antibiotic gel and injected into the joint capsule. This treats the infection and counteracts damage to articular cartilage. Inclusion of anti-inflammatory agents is also useful.

Delivery of osteoblasts is advantageous when an orthopedic defect is present. An anti-infective sterilizes the site and the osteoblasts facilitate osteogenesis. Various cytokines and osteogenic proteins can optionally be incorporated.

The following Examples are illustrative, but not limiting of the compositions and methods of the present invention. Other suitable modifications and adaptations of a variety of conditions and parameters normally encountered which are obvious to those skilled in the art are within the spirit and scope of this invention.

### EXAMPLES

### Example 1

Preparation of Dextran Sulfate/Amikacin

The sodium salt of dextran sulfate (Mw 500,000, 450 mg) was dissolved in a minimum amount of water (about one ml). Amikacin sulfate (780 mg), dissolved in a minimum amount of water (about 2 ml) was added to the dextran sulfate solution and mixed thoroughly at room temperature. After about 5 minutes of spatulation, the supernatant (about 40% of original volume) was poured off and the viscous gel was collected and stored at room temperature, protected from light.

Release profile: Dextran sulfate/amikacin wet gel (100 mg) was placed in 2 ml centrifuge tube. PBS buffer (500 μl) was added to the centrifuged tube. After incubation at 37° C. for 24 hrs, the mixture was centrifuged at 13,000 RPM for 5 minutes. The supernatant was removed and analyzed microbiologically for the presence of drug. The process was repeated at 24 hr intervals for 31 days. The amount of released drug in the eluate was calculated from a standard curve.

The release profile is illustrated in FIG. 1. The release profiles for the compounds of the other Figures were generated in a similar manner.

### Example 2

Preparation of Dextran Sulfate/Vancomycin

The sodium salt of dextran sulfate (Mw, 500,000) (100 mg) was dissolved in a minimum amount of water (about 0.5 ml). Vancomycin hydrochloride (165 mg) was also dissolved in minimum amount of water (about 0.5 ml). The solutions were mixed at room temperature and stirred with a spatula for 5 minutes. The resulting gel, which constituted the entire mixture, was centrifuged at 12,000 rpm for 5 min. The supernatant (about 30% of original volume) was removed from centrifuge tube. The gel was air dried for 48 hrs and then finely ground. The release profile is shown in FIG. 1.

### Example 3

Preparation of Dextran Sulfate/Gentamicin

The sodium salt of dextran sulfate (Mw 500,000; 300 mg) was dissolved in a minimum amount of water (about 0.8 ml). Gentamicin sulfate (110 mg) dissolved in about 0.5 ml of water, was added to the dextran sulfate solution and mixed thoroughly at room temperature with spatulation. After about 5 minutes of mixing the supernatant (about 40% of original volume) was poured off and the viscous gel was collected and stored at room temperature, protected from light.

### Example 4

Preparation of Dextran Sulfate/Clindamycin

The sodium salt of dextran sulfate (500,000 Mw; 110 mg) was dissolved in a minimum amount of water (about 0.5 ml). Clindamycin-HCl (230 mg), dissolved in a minimum amount of water (about 0.5 ml) was added to the dextran sulfate solution and mixed thoroughly at room temperature. After about 5 minutes of spatulation, the supernatant (about 50% of original volume) was poured off and the gummy complex was collected and stored at room temperature, protected from light.

### Example 5

Preparation of Dextran Sulfate/Doxycyline

The sodium salt of dextran sulfate (500,000 Mw; 225 mg) was dissolved in a minimum amount of water (about 0.7 ml). Doxycycline hydrochloride (120 mg) was also dissolved in minimum amount of water (about 0.5 ml). The solutions were mixed at room temperature and stirred with a spatula for 5 minutes. The resulting gel, which constituted the entire mixture, was air dried for 48 hrs and then finely ground.

### Example 6

Preparation of Dextran Sulfate/Hydromorphone

The sodium salt of dextran sulfate (Mw; 500,000, 75 mg) was dissolved in a minimum amount of water (about 0.3 ml). Hydromorphone hydrochloride (110 mg), dissolved in minimum amount of water (about 0.3 ml) was added to the dextran sulfate solution and mixed thoroughly at room temperature. After about 5 minutes of spatulation, the supernatant (about 50% of original volume) was poured off and the gummy complex was air dried for 48 hrs and then finely ground.

### Example 7

Preparation of Dextran Sulfate/Dibucaine

The sodium salt of dextran sulfate (Mw 500,000; 150 mg) was dissolved in a minimum amount of water (about 0.3 ml). Dibucaine hydrochloride (130 mg), dissolved in minimum amount of water (about 0.4 ml), was added to the dextran sulfate solution. The solutions were mixed at room temperature and stirred with a spatula for 5 minutes. The supernatant (about 40% of original volume) was removed. The resulting viscous complex was air dried for 48 hrs and then finely ground.

### Example 8

Preparation of Dextran Sulfate/Tetracaine

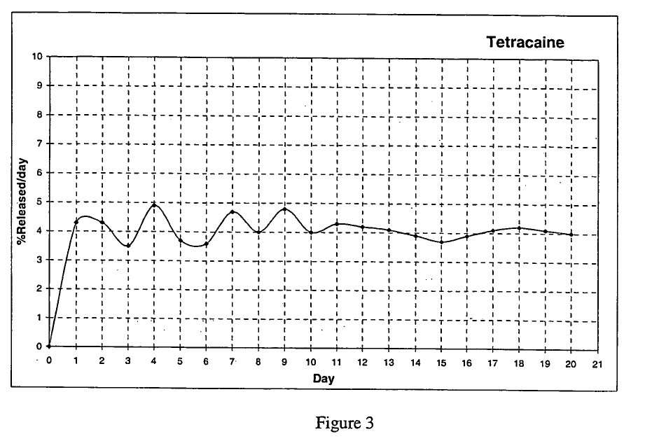

The sodium salt of dextran sulfate (Mw 500,000; 75 mg) was dissolved in a minimum amount of water (about 0.25 ml). Tetracaine-HCl (100 mg), also dissolved in minimum amount of water (about 0.5 ml), was added to the dextran sulfate solution and mixed thoroughly at room temperature. After about 5 minutes of spatulation, the supernatant (about 70% of the original volume) was poured off and the gummy complex was air dried for 48 hrs and then finely ground. The release profile is shown in FIG. 3.

### Example 9

Preparation of Carboxymethylcellulose/Dibucaine

The sodium salt of carboxymethylcellulose, medium or low viscosity (CMC-M or CMC-L, 80 mg) was dissolved in about 0.8 ml of water. Dibucaine hydrochloride (130 mg) dissolved in minimum amount of water (about 0.25 ml) was added to the carboxymethylcellulose solution and mixed thoroughly at room temperature. After about 5 minutes stirring with a spatula, the supernatant (about 40% of the original volume) was poured off and the viscous gel was collected and stored at room temperature, protected from light.

### Example 10

Preparation of Carboxymethylcellulose/Tetracaine

CMC-M or CMC-L (80 mg in each case) was dissolved in 0.8 ml of water. Dibucaine hydrochloride (100 mg), dissolved in a minimum amount of water (about 0.5 ml), was added to the carboxymethylcellulose solution and mixed thoroughly at room temperature. After about 5 minutes stirring with a spatula, the supernatant (about 60% of original volume) was poured off and the viscous gel was collected and stored at room temperature, protected from light.

### Example 11

Preparation of Carboxymethylcellulose/Doxycycline

CMC-M or CMC-L (80 mg) was dissolved in 0.8 ml of water. Doxycycline hydrochloride (160 mg), dissolved in minimum amount of water (about 0.5 ml), was added to the carboxymethylcellulose solution and mixed thoroughly at room temperature. After about 5 minutes of spatulation, the supernatant (about 50% of original volume) was poured off and the residual complex was air dried for 48 hrs and then finely ground.

### Example 12

Preparation of Carboxymethylcellulose/Vancomycin

CMC-M or CMC-L (50 mg) was dissolved in 0.5 ml of water. Vancomycin hydrochloride (160 mg) was also dissolved in minimum amount of water (about 0.5 ml). The solutions were mixed at room temperature and stirred with a spatula for 5 minutes. The resulting gel, which constituted the entire mixture, was centrifuged at 12,000 rpm for 5 min. The supernatant (about 40% of original volume) was removed and discarded. The gel was air dried for 48 hrs and then finely ground.

### Example 13

Preparation of Amikacin Cylinders

Calcium sulfate/calcium stearate (95/5 wt/wt, 300 mg) was mixed with 300 mg of amikacin gel (dextran sulfate/amikacin). After about 1 minute of stirring the resulting slurry was transferred to the barrel of a 3 ml syringe. Then the slurry was injected into a silicone rubber mold with cylindrical holes (length 3 mm; diameter 4 mm). After 24 hours at room temperature, the cylinders were removed from mold.

### Example 14

Preparation of Amikacin Gum

Amikacin gel (dextran sulfate/amikacin, 200 mg) was mixed with 200 mg calcium stearate. To this mixture 200 mg of the calcium sulfate dihydrate was added. After mixing for one minute, an additional 100 mg of the calcium sulfate dihydrate was added and the mass was kneaded by hand for about 2 minutes. Advantageously, the gum is formed and installed in an orthopedic defect within one hour. The gum can be stored in an airtight container at 0-4 C for at least two weeks.

### Example 15

Preparation of Doxycycline Complex Cement and Microgranules

Doxycycline complex (dried dextran sulfate/doxycyline, 250 mg) was finely ground and mixed with 3.5 g of calcium sulfate hemihydrate/calcium stearate (95/5, wt/wt). To this mixture 2.8 ml of the water for injection was added with mixing. The resulting slurry was poured into a tray and allowed to solidify. The solid was milled and sized to 45-150 microns. Alternatively, the slurry can be injected directly into an orthopedic/periodontal defect.

### Example 16

Demonstration of Sustained Release of Amikacin in an Animal

Amikacin gel (1 ml) prepared as described in Example 1 was injected into the hock joint of a horse which was prepped by shaving and treatment with povidone-iodine. Samples of synovial fluid were taken at timed intervals and the levels of amikacin were determined using an immunofluorescent assay system. Results appear in Table 2.

TABLE 2
In vivo levels of drug following intra-articular
injection of amikacin gel.

| Time | Amikacin Levels |
|---|---|
| [Days Elapsed Post injection] | [μg/ml] |
| 1 | 224.85 |
| 2 | 54.8 |
| 3 | 4.81 |
| 4 | 3.35 |
| 5 | 1.9 |
| 6 | 0.44 |

Depending on the target organism, therapeutic levels are maintained for at least 5 days. Some MICs (minimum inhibitory concentration) are shown below for amikacin:

| Organism | MIC (amikacin, ug/ml) |
|---|---|
| S. Aureus | 1 |
| E. Coli | 2 |
| Enterobacter spp. | 2 |
| P. Aeruginosa | 2 |

### Example 17

Preparation of Dextran Sulfate/Oxycodone

The sodium salt of dextran sulfate (Mw; 500,000, 50 mg) was dissolved in a minimum amount of water (about 0.25 ml). Oxycodone hydrochloride (78 mg), dissolved in minimum amount of water (about 0.5 ml) was added to the dextran sulfate solution and mixed thoroughly at room temperature. After about 5 minutes of spatulation, the supernatant (about 75% of original volume) was poured off and the gummy complex was air dried for 48 hrs and then finely ground.

The sodium salt of high molecular weight dextran sulfate (Mw; 500,000, 25 mg) plus the sodium salt of low molecular weight sulfate (Mw; 40,000-50,000, 25 mg) were mixed and dissolved in a minimum amount of water (about 0.25 ml). Oxycodone hydrochloride (78 mg), dissolved in minimum amount of water (about 0.5 ml) was added to the dextran sulfate solution and mixed thoroughly at room temperature. After about 5 minutes of spatulation, the supernatant (about 79% of original volume) was poured off and the viscous product was air dried for 48 hrs and then finely ground. The release profiles are shown in FIG. 6. The inclusion of low molecular weight polymer increases the release rate.

### Example 18

Combination Product—Liquid/Liquid:Amikacin/Vancomycin

Dextran sulfate/amikacin gel (500 mg, Example 1) was mixed with an equivalent amount of dextran sulfate/vancomycin gel (Example 2). The product mixture was even more viscous than the starting materials. A supernatant (about 30% of the original volume) was decanted. The product mixture was stored in the dark at room temperature. Installation of this product is best done with a syringe without a needle or a syringe fitted with a large cannula.

### Example 19

Suspension Product—Dextran Sulfate Vancomycin (Dry) in Dextran Sulfate (Liquid)

Dextran sulfate, sodium salt (Mw=500,000; 225 mg) was dissolved in 0.5 ml distilled water. Dextran sulfate-vancomycin complex (dry, finely ground, 150 mg) prepared as described in Example 2, was added to the polymer solution and mixed for 5 minute with a spatula. The mixture was stored at room temperature in the dark. This product was injectable through an 18-gauge needle. A similar product can be made starting with a CMC solution, namely 25 mg CMC-M in 0.5 ml distilled water.

### Example 20

Suspension Product: Enrofloxacin-HCL in Dextran Sulfate (Sodium) Solution

Dextran sulfate (sodium salt, Mw=500,000, 900 mg) was dissolved in 2 ml of distilled water. Enrofloxacin-HCL powder (800 mg) was added to the dextran sulfate solution and mixed for 15 minutes at room temperature. The product was stored at room temperature in the dark and is injectable through a 20-gauge needle.

### Example 21

Suspension Product: Bupivacaine Salts in Dextran Sulfate (Sodium) Solution

Bupivacaine pamoate (100 mg) and bupivacaine-HCL (100 mg) were ground together with a mortar and pestle. Dextran sulfate solution (as above, 0.34 ml) was added and the suspension was mixed for 15 minutes at room temperature. The suspension was stored in a syringe at room temperature in the dark.

### Example 22

Inclusion Product: Ivermectin in Dextran Sulfate-Amikacin

Ivermectin (300 mg) was finely ground and suspended in 0.5 ml of dextran sulfate solution (sodium salt, 45% w/v). Finely ground amikacin sulfate (100 mg) was added and the mixture was processed for 3 minutes with a mortar and pestle. The product was stored at room temperature in the dark and was easily syringable through a 20-gauge needle.

It will be readily apparent to those skilled in the art that numerous modifications and additions may be made to the present invention, the disclosed device, and the related system without departing from the invention disclosed.

What is claimed is:

1. A sustained release composition comprising a cationic active agent, and a polyanionic water-soluble complexing polymer of sufficient molecular weight that it forms a gel when mixed with said active agent.

2. A composition as in claim 1 further comprising calcium sulfate.

3. A composition as in claim 1 further comprising a mixture of phosphates, which includes tricalcium phosphate.

4. A composition as in claim 1 further comprising hydroxyl apatite.

5. A composition as in claim 1 wherein said complexing polymer is at least two complexing polymers of different molecular weight.

6. A composition as in claim 1 wherein said active agent is an anti-infective selected from the group consisting of gentamicin, azithromycin, clarithromycin, doxycycline, minocycline and lincomycin, clindamycin, amikacin, vancomycin, tobramycin, nystatin, and amphotericin B.

7. A composition as in claim 1 wherein said active agent is an anesthetic selected from the group consisting of dibucaine, tetracaine, procaine, etidocaine, bupivacaine, mepivacaine, and prilocaine.

8. A composition as in claim 1 wherein said active agent is an opioid/analgesic selected from the group consisting of fentanyl, sufentenil, morphine, methadone, etorphine, levorphanol, levallorphan, butorphenol, buprenorphine, oxycodone, hydromorphone, propoxyphene, naloxone, naltrexone, nalorphine, nalbuphine, nalmefene, codeine, oxymorphone, and dermorphine.

9. A composition as in claim 1 wherein said active agent is an anti-tumor agent.

10. A composition as in claim 1 wherein said active agent is a CNS agent selected from the group consisting of acepromezine, prochlorperazine, clomipramine, ondansetron, sertraline, doxazosine, chlorpromazine, and atropine.

11. A composition as in claim 1 wherein said complexing polymer is selected from the group consisting of dextran sulfate, carboxymethylcellulose, and pentosan sulfate.

12. A composition as in claim 1 wherein said complexing polymer is dextran sulfate (Na).

13. A composition as in claim 12 wherein said complexing polymer is dextran sulfate (Na) of molecular weight 500,000 or higher.

14. A composition as in claim 1 wherein said complexing polymer is carboxymethylcellulose.

15. A composition as in claim 1 wherein said complexing polymer is L-carboxymethylcellulose.

16. A composition as in claim 1 wherein said complexing polymer is M-carboxymethylcellulose.

17. A method of treating an infection in a mammal comprising administering to said mammal a sustained release composition comprising a cationic anti-infective and a polyanionic water soluble complexing polymer of sufficient molecular weight that it forms a gel with said anti-infective.

18. A method of treating bone sepsis, joint sepsis, an infected joint prosthesis, a diabetic foot infection, or periodontal disease, in a mammal comprising administering by injection to said mammal a composition comprising active agent selected from the group consisting of gentamicin, azithromycin, clarithromycin, doxycycline, minocycline and lincomycin, clindamycin, amikacin, vancomycin, tobramycin, nystatin, and amphotericin B. and a complexing polymer of sufficient molecular weight that it forms a gel with said anti-infective.

19. A method of systemically treating an infection in a mammal comprising administering subcutaneously to said mammal a composition comprising active agent selected from the group consisting of gentamicin, azithromycin, clarithromycin, doxycycline, minocycline and lincomycin, clindamycin, amikacin, vancomycin, tobramycin, nystatin, and amphotericin B and a complexing polymer.

20. A method of regionally blocking nerves or treating localized pain in a mammal comprising administering by injection to said mammal a composition comprising an anesthetic selected from the group consisting of dibucaine, tetracaine, procaine, prilocaine, etidocaine, bupivacaine, mepivacaine, and a complexing polymer.

21. A method of treating pain in a mammal comprising administering to said mammal a composition comprising an active agent selected from the group consisting of oxycodone, morphine, fentanyl, sufentanil, and hydromorphone, and a complexing polymer.

22. A method as in claim 21 wherein said complexing polymer is at least two complexing polymers of different molecular weight.

23. A method of treating drug addiction in a mammal comprising administering to said mammal a composition comprising an active agent selected from the group consisting of methadone, buprenorphine, naloxone, and naltrexone, and a complexing polymer.

24. A method of treating cancer in a mammal comprising administering to said mammal a sustained release composition comprising a cationic anti-tumor agent and a polyanionic water soluble complexing polymer.

25. A molded prosthesis comprising a prosthesis including a sustained release composition comprising a cationic active agent selected from the group consisting of gentamicin, azithromycin, clarithromycin, doxycycline, minocycline and lincomycin, clindamycin, amikacin, vancomycin, tobramycin, nystatin, and amphotericin B., and a polyanionic water soluble complexing polymer of sufficient molecular weight that it forms a gel with said active agent.

26. A method of producing a sustained release gel composition comprising mixing a cationic active agent and a polyanionic water soluble complexing polymer of sufficient molecular weight that it forms a gel with said active agent.

27. A method of producing a sustained release composition comprising mixing a cationic active agent and a polyanionic water soluble complexing polymer of sufficient molecular weight that it forms a gel with said active agent, drying the gel, grinding the dried gel to a powder, and suspending the powder in a suspending agent.

28. A method of producing a sustained release composition comprising mixing a cationic active agent and a polyanionic water soluble complexing polymer of sufficient molecular weight that it forms a gel with said active agent, and adding calcium sulfate to the gel.

29. A method as in claim 28 wherein the calcium sulfate is calcium sulfate hemihydrate.

30. A method as in claim 28 wherein the calcium sulfate is calcium sulfate dihydrate.

31. A composition comprising a mixture of at least two polymer-drug complexes each of which contains a distinct active ingredient.

32. A composition comprising a solid polymer drug complex suspended in a liquid polymer-drug complex.

33. A composition comprising a polymeric anion with a poorly soluble cationic drug complex of low molecular weight.

34. A composition comprising a neutral drug entrapped within a cross-linked reaction product of a polymeric anion and a cation cross-linking agent.
